(19) United States
(12) Patent Application Publication
(10) Pub. No.: US 2014/0316222 A1
(43) Pub. Date: Oct. 23, 2014

(54) IN-VIVO DEVICE, SYSTEM AND METHOD FOR DETECTION OF HELICOBACTER PYLORI

(71) Applicant: GIVEN IMAGING LTD., Yoqneam (IL)

(72) Inventors: Elisha RABINOVITZ, Haifa (IL); Osnat Sella-Tavor, Kfar Kish (IL)

(73) Assignee: GIVEN IMAGING LTD., Yoqneam (IL)

(21) Appl. No.: 14/259,946

(22) Filed: Apr. 23, 2014

Related U.S. Application Data
(60) Provisional application No. 61/815,022, filed on Apr. 23, 2013.

(51) Int. Cl. A61B 5/07; A61B 5/053; A61B 5/00

(52) U.S. Cl. CPC A61B 5/073; A61B 5/0084; A61B 5/0538; A61B 5/742; A61B 5/42
USPC 600/302

(57) ABSTRACT

Device, system and method for in-vivo detection of H. pylori. For example, an in-vivo device includes a housing, a reference electrode disposed on the housing, and a working electrode disposed on the housing in close proximity to the reference electrode. The working electrode is coated by litography or screen printing with salt such that substantially no current passes between the working electrode and the reference electrode. The in-vivo device further includes a measuring device for measuring current, impedance, and/or resistance between the working and the reference electrodes, and a transmitter for transmitting the measurements. Presence of ammonia, which is a byproduct of H. pylori, causes increase in current and thus decrease in impedance or resistance between the reference electrode and the working electrode; thereby measurements of current, impedance, and/or resistance indicate on presence or absence of H. pylori.

### TECHNICAL FIELD

This application relates to the field of in-vivo devices, systems, and methods of in-vivo detection of Helicobacter pylori.

### BACKGROUND

Helicobacter pylori (H. pylori) is a bacterium residing in the mucus lining of the stomach and the duodenum. It was initially found in patients who suffered from chronic gastritis and gastric ulcers. More than 50% of the world's population harbor H. pylori in their upper gastrointestinal (GI) tract, though may not be infected. Patients who are infected with H. pylori are in risk of developing peptic ulcers and even stomach cancer.

H. pylori bacterium produces large amounts of the enzyme urease, which breaks down urea (normally secreted into the stomach) to carbon dioxide and ammonia. Ammonia is toxic to the epithelial cells of the stomach, thus ammonia damages those cells, which may lead to ulcers inside the stomach appearing as abdominal pain and/or nausea.

A few of the methods used for the detection of H. pylori include non-invasive methods, e.g., breath test, which detects the amount of carbon dioxide labeled with uncommon carbon isotope, either radioactive carbon-14 or non-radioactive carbon-13, exhaled by the patient (following consumption of urea labeled with uncommon carbon isotope), blood antibody test for detecting antibodies for H. pylori in the patient's blood, or stool antigen test. An invasive method of detection of H. pylori includes a biopsy performed during endoscopy with rapid urease test, histological examination, and microbial culture. However, none of these methods are completely failsafe, even the invasive method, which is obviously very unpleasant for the patient undergoing it.

There is therefore a need for a user-friendly and yet reliable in-vivo device, system and method for detecting presence of H. pylori in the stomach or gastric fluids of a patient.

### SUMMARY

The present invention provides in-vivo devices, systems, and methods for detection of H. pylori. An in-vivo device for in-vivo detection of H. pylori, according to some embodiments, may comprise a housing, a reference electrode and a working electrode both disposed on the housing in close proximity to one another. According to some embodiments, the reference electrode may be made of inert material, e.g., gold, and the working electrode, which may also be made of inert material, is coated by lithography or screen printing with coating containing salt that dissolves in the presence of ammonia, e.g., silver-chloride salt, such that substantially no or nearly no current passes between the working electrode and the reference electrode in initial state, namely—when coated. The device may further comprise a device for measuring current, impedance, and/or resistance between the working and the reference electrodes. The device for measuring current may be an ampere-meter. In the presence of H. pylori, the salt coating may dissolve and a current increase and/or a resistance decrease (when constant voltage is applied in the device) may be measured. The device may further comprise a transmitter for transmitting the measurements of current, impedance, and/or resistance. A processor may process the measurement such to provide an indication to the absence or presence of ammonia and therefore, of the absence or presence of H. pylori. In some embodiments, the device may comprise an imager for acquiring in-vivo images. The in-vivo device may be an autonomous swallowable device.

A system for in-vivo detection of H. pylori may comprise the in-vivo device for in-vivo detection of H. pylori, which may comprise the two electrodes and the device for measuring current, impedance, and/or resistance as described above. The system may further comprise a receiver for receiving the transmitted measurements of current, impedance, and/or resistance. The system may further comprise a processing unit for processing the received current, impedance, and/or resistance measurements and determining presence of H. pylori, and a display unit for displaying the current, impedance, and/or resistance measurements with or without the determination on presence of H. pylori.

Another in-vivo device for in-vivo detection of H. pylori, may comprise a housing, a gap through which in-vivo fluids may enter and exit, and a mixture of substrate and enzyme for reacting with ammonia, or a mixture of substrate and urea reacting with the urease, which causes the substrate to change its optical or electrical properties. The device may comprise a gap and may further comprise an illumination source for illuminating the mixture. The illumination source may be located on one side of the gap, and a detector for detecting the change in optical properties of the substrate which indicates in-vivo presence of H. pylori, may be located on the opposite side of the gap, and facing across the gap towards the illumination source. The device may further comprise a transmitter for transmitting the detected changes in optical properties indicating presence of H. pylori. The device may be an autonomous swallowable device. According to some embodiments, the change in optical property of the substrate is a change in light absorbance by the substrate.

A system for in-vivo detection of H. pylori may comprise the device for in-vivo detection of H. pylori, which may comprise the gap and mixture (described above), and a receiver for receiving the transmitted changes in optical properties. The system may further comprise a processing unit for processing the received changes in optical properties and determining presence of H. pylori, and a display unit for displaying the changes in optical properties with without the determination on presence of H. pylori.

A method for in-vivo detection of H. pylori may comprise the following steps of:
administering into gastric fluids a swallowable in-vivo device comprising a reference electrode, a working electrode coated by lithography or screen printing with urea and/or salt that dissolves in presence of ammonia;
applying constant voltage between the two electrodes,
measuring current, impedance and/or resistance between the two electrodes; and
determining in-vivo presence of H. pylori in-vivo based on the change in current, impedance and/or resistance measurements.

According to some embodiments, an increase in current measurements between the two electrodes (or a decrease in impedance and/or resistance measurements between the electrodes) indicates presence of H. pylori in-vivo.

Another method for in-vivo detection of H. pylori may comprise the following steps:
administering in-vivo a swallowable device, said device comprising: a gap, an illumination source on one side of the gap, a detector on the opposite side of the gap and facing across the gap towards the illumination source, and a substrate and enzyme located within the gap for detection of ammonia,
allowing flow of in-vivo fluids through the gap, and thus contact between the substrate, enzyme, and ammonia; and
determining in-vivo presence or absence of H. pylori, based on change in optical properties of the substrate.

### BRIEF DESCRIPTION OF THE DRAWINGS

The above and other objects and advantages of the invention will be apparent upon consideration of the following detailed description, taken in conjunction with the accompanying drawings, in which the reference characters refer to like parts throughout and in which:

FIG. 1A illustrates a schematic side view of an in-vivo device and system, in accordance with an embodiment of the present invention;

FIG. 1B illustrates a schematic side view of an in-vivo device, in accordance with an embodiment of the present invention;

FIG. 1C illustrates a schematic side view of an in-vivo device, in accordance with an embodiment of the present invention;

FIG. 2 illustrates a method of in-vivo detection of H. pylori, in accordance with an embodiment of the present invention;

FIG. 3 illustrates a method of in-vivo detection of H. pylori, in accordance with another embodiment of the present invention;

FIG. 4 illustrates a schematic side view of an in-vivo device and system, in accordance with an embodiment of the present invention

FIG. 5 illustrates a schematic front-side view of an in-vivo device and system, in accordance with an embodiment of the present invention; and

FIG. 6 illustrates a method of in-vivo detection of H. pylori, in accordance with an embodiment of the present invention.

It will be appreciated that for simplicity and clarity of illustration, elements shown in the figures have not necessarily been drawn to scale. For example, the dimensions of some elements may be exaggerated relative to other elements for clarity. Further, where considered appropriate, reference numerals may be repeated among the figures to indicate corresponding or analogous elements.

### DETAILED DESCRIPTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

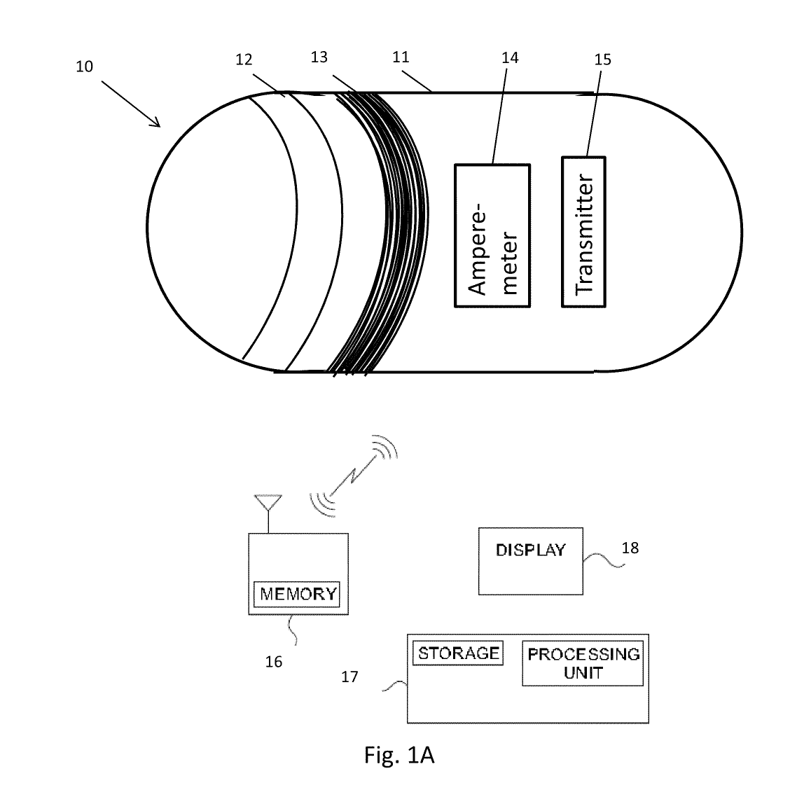

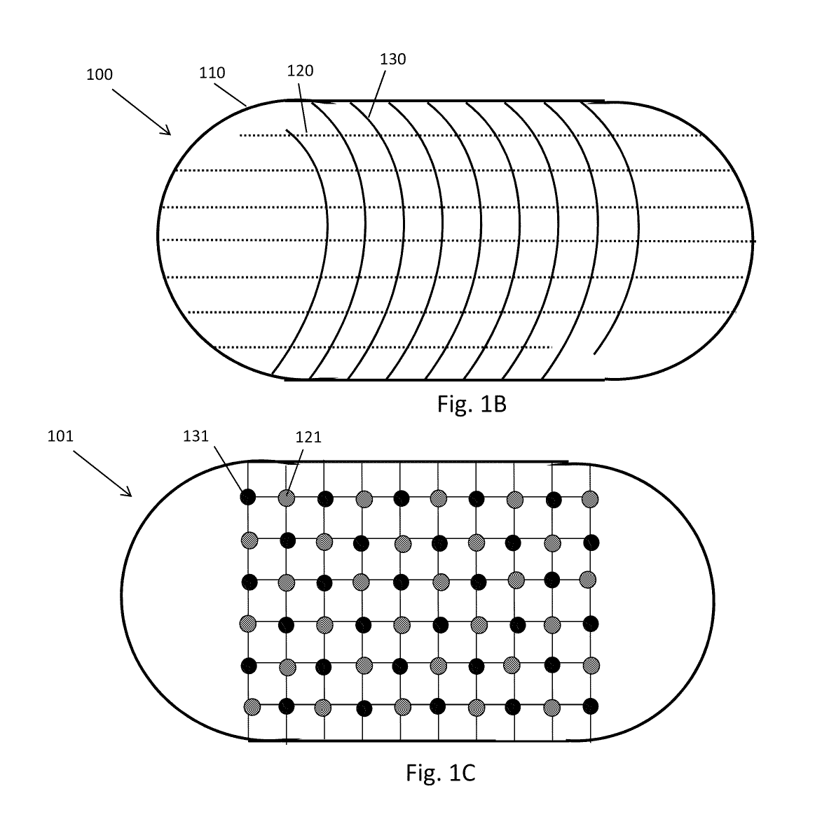

Reference is now made to FIG. 1, which illustrates a schematic side view of an in-vivo device and system for detection of H. pylori, in accordance with an embodiment of the present invention. Device 10 may be an autonomous swallowable device. Device 10 may be in the size of between several millimeters and up to about 13 mm in diameter. Device 10 is typically made of biocompatible materials, which are also electrically inert, e.g., polycarbonate. Device 10 may comprise housing 11, which may have various shapes, e.g., capsule, spherical, ellipsoidal, etc. Device 10 may comprise a reference electrode 12, which may be disposed on the external surface of housing 11 or may otherwise be fully or partially exposed to the outside of housing 11. In some embodiments, reference electrode 12 may be made of an inert material, e.g., gold. In some embodiments, electrode 12 may be printed onto housing 11. As shown in FIG. 1A, reference electrode 12 may be disposed at one of the ends of device 10 in the shape of a ring, which surrounds the external surface of housing 11, though any other shape in any other location along housing 11 is possible. Device 10 may comprise an additional electrode, which may be disposed on or printed on housing 11 at a distance from reference electrode 12 or may otherwise be fully or partially exposed to the outside of housing 11, for example, working electrode 13. Typically, working electrode 13 may be in close proximity to, but at a distance from, reference electrode 12. In some embodiments, working electrode 13 may be disposed on the same end of elongated housing 11 as reference electrode 12, though other locations along housing 11 are possible. In some embodiments, as shown in FIG. 1A, electrode 13 may be in the shape of a ring surrounding the external surface of housing 11, though other shapes may be possible.

As long as there is an electrically insulated area between the two electrodes, on the external surface of housing 11, the distance between electrode 12 and electrode 13 may be as small as a few microns. However, the distance between the electrodes may reach the distance of a few centimeters, (almost equal to the entire length of device 10), e.g., when one electrode is located at one end of elongated housing 11, and the other electrode is located at the opposite end of elongated housing 11.

In some embodiments, working electrode 13 may be made of an inert material, e.g., gold. In some embodiments, working electrode 13 may be covered with a layer of coating mixture by litography means or screen printing. The coating mixture may comprise urea and/or a salt that dissolves in the presence of ammonia, and a solvent. The solvent is mixed with the salt when the solvent is in liquid state, in order to enable litography or screen printing coating of electrode 13. However, after the solvent is dried or polymerized it changes its characteristics, and becomes a binder that may create a non-porous layer or surface covering electrode 13. The ratio between concentration of the salt and that of the solvent affect the level of permeability of the coated layer to ammonia (and not to other in-vivo fluids). That is, the more salt used compared to the amount of solvent used to create the coating mixture, the more permeable the layer is to ammonia. On the contrary, the more solvent used compared to the amount of salt used to create the coating mixture; the more impermeable the coated layer is to ammonia. Permeability to ammonia of the coated layer affects time till identification of presence of ammonia is made, which also affects sensitivity of the coated layer to presence of ammonia. The more permeable the coated layer is, the less time would elapse before determining presence of ammonia and thus presence of H. pylori. This also means that the more permeable the coated layer is, the more sensitive it is to low concentrations of ammonia. And vice versa, the more impermeable the coated layer is, the more time would pass before determining presence of ammonia, which also means that the more impermeable the coated layer is, the less sensitive it is to low concentrations of ammonia. The ratio between concentrations of the salt and solvent may be chosen based on the above considerations such to create a semi-permeable coated layer covering working electrode 13.

The minimum concentration of ammonia that should be detected by the device is 1 milimole per liter, which is 1 micromole per milileter. There are two ways by which it is possible to increase sensitivity of working electrode 13 to such a low concentration of ammonia, while decreasing the time till an increase in current is measured. One way is by increasing the amount of salt compared to that of the solvent, both of which comprise the coating, i.e., more salt is used in the coating compared to the solvent, and another way is by covering electrode 13 with a thin layer of coating.

Litography is one example for methods of coating that enable use of a solvent/binder, which creates a non-porous surface or layer. Methods that do not involve use of such solvent/binder, e.g., electro-chemical coating methods, and most of the evaporating methods, result in porous surfaces or layers, which enable transfer of ions through the coated layer and thus are not appropriate for the present invention. Methods that do not involve use of a solvent/binder or polymerization step do not enable good control of the level of permeability of the coated layer, as possible in lithography, screen printing and other methods of coating, which involve a solvent/binder or polymerization. In litography, as well as in methods involving a solvent/binder, the degree of permeability may be designed by choosing the ratio between the salt and the solvent or the filler and the polymer. Methods that do not involve a way to affect the degree of permeability of the coated layer are methods unsuitable for the present invention.

In some embodiments, the salt, which the coating is partially composed of, may be silver-chloride salt, though other salts may be used. In other embodiments, urea is used and the decomposition of the urea to ammonia is done by the urease enzyme produced by the H. pylori. A mixture of urea and silver-chloride salt may be used to enhance the dissolution process. The solvent may, for example, be linolenic acid, though other solvents may be used. Following the litography process, a semi-permeable layer covers working electrode 13. When the coated layer on working electrode 13 is intact, e.g., in the absence of ammonia, it acts to isolate the electrode from its environment (e.g., from in-vivo fluids), and causes a short circuit, thus substantially no current passes between the working electrode 13 and reference electrode 12. Substantially no current passing between the two electrodes means that current that might be shown to pass between the electrodes is smaller than the measurement error of the device measuring the current. If ammonia is present in the stomach, it may pass through the semi-permeable coated layer covering electrode 13 and dissolve the salt within the coated layer, thus penetrating through the coated layer towards working electrode 13. Once the coated layer is compromised, current may pass between working electrode 13 and reference electrode 12 (i.e., there is an increase in current), and measurements of such increase in current may indicate presence of ammonia, thus indicate presence of H. pylori.

Device 10 may further comprise a measuring device 14, e.g., an ampere-meter, for measuring current, impedance and/or resistance between reference electrode 12 and working electrode 13, and a transmitter 15 for transmitting the measured current to an external receiver 16. Typically, transmission of the measured data (e.g., current measurements) is done wirelessly, e.g., by RF transmission. According to some embodiments, a system for in-vivo detection of H. pylori may comprise device 10, external receiver 16, storage and processing unit 17 and display unit 18. Receiver 16 may comprise a memory unit to store all measured data. In some embodiments, receiver 16 may further comprise storage and processing unit 17 for processing the measured data and for providing an indication of presence or absence of H. pylori within the gastric fluids. However, in other embodiments, storage and processing unit 17 may be a unit separate from receiver 16. The measured data and/or determination of presence or absence of H. pylori in-vivo may be displayed on display unit 18, which may, for example, be a computer or TV screen. In some embodiments, communication between receiver 16, processing unit 17 and display unit 18 may be wireless, though in other embodiments communication between those components may be wired.

In some embodiments, ampere-meter 14 may measure current, which may be translated into resistance by using the simple equation (i) V=IR. Since constant voltage (V) is applied in device 10, and current (I) is measured by ampere-meter 14, then resistance (R) may be easily calculated by, for example, processing unit 17. In other embodiments, impedance may be measured. Transmitter 15 may then transmit current measurements, impedance measurements and/or resistance measurements to an external receiver, e.g., receiver 16. In other embodiments, an ohmmeter may replace ampere-meter 14 in order to directly measure resistance.

In the presence of ammonia, when constant voltage is applied in device 10, current increases (since ammonia causes contact between working electrode 13 and in-vivo fluids) and thus resistance decreases, as apparent from equation (i).

Reference is now made to FIG. 1B, which illustrates a schematic side view of an in-vivo device for detection of H. pylori, in accordance with an embodiment of the present invention. In this second embodiment, in-vivo device 100 may have a shape that may be easily inserted into a patient's GI tract, e.g., by swallowing. For example, device 100 may be in the shape of a capsule, though other shapes are possible. Device 100 may comprise a reference electrode 120, which may surround the external surface of housing 110 of device 100. Reference electrode 120 may be twisted around the external surface of housing 110 in a snake-like form. Reference electrode 120 may be twisted around housing 110 along one of the axes of device 100, e.g., along the longitudinal axis of device 100. According to FIG. 1B, device 100 may further comprise working electrode 130, which may also surround the external surface of housing 110 of device 100. However, typically, working electrode 130 may be twisted around a different axis of device 100 than that surrounded by reference electrode 120. For example, if reference electrode 120 is twisted around the longitudinal axis, then working electrode 130 is twisted around the lateral axis of device 100. The advantage of these ‘snake-like’ shaped electrodes is that such a shape of electrodes surrounding device 100 increases the area of working electrode 130 that may be in contact with ammonia, which if present indicates in-vivo presence of H. pylori. When the electrodes nearly surround the entire external surface of housing 110, there is more chance of device 100 to detect presence of ammonia, as there is more chance that ammonia would be in contact with housing 110, and thus in contact with at least part of working electrode 130.

Reference is now made to FIG. 1C, which illustrates a schematic side view of an in-vivo device for detection of H. pylori, in accordance with another embodiment of the present invention. According to FIG. 1C, device 101 may comprise an array of dot shaped reference electrodes 121 and dot shaped working electrodes 131. The two types of electrodes may be positioned in an alternately arrangement, e.g., that each reference electrode 121 has working electrodes 131 as its neighbor electrodes, e.g., from all four sides of reference electrode 121 (i.e., up, down, left and right), and vice versa.

Device 101 may have the advantage of working and reference electrodes 131 and 121, respectively, substantially surrounding the entire external surface of the housing of device 101, which increases the chance of device 101 to detect presence of ammonia, as explained above with regards to FIG. 1B. However, contrary to device 100 (FIG. 1B), which includes one input and one output per each electrode (whether the reference electrode 120 or the working electrode 130), device 101 need many more inputs and outputs since it comprises many more reference and working electrodes. Each of the dot shaped electrodes surrounding device 101 need one input and one output, which makes device 101 more complex to manufacture than device 100, since device 101 comprises more than one reference electrode 121 and more than one working electrode 131, as in device 100.

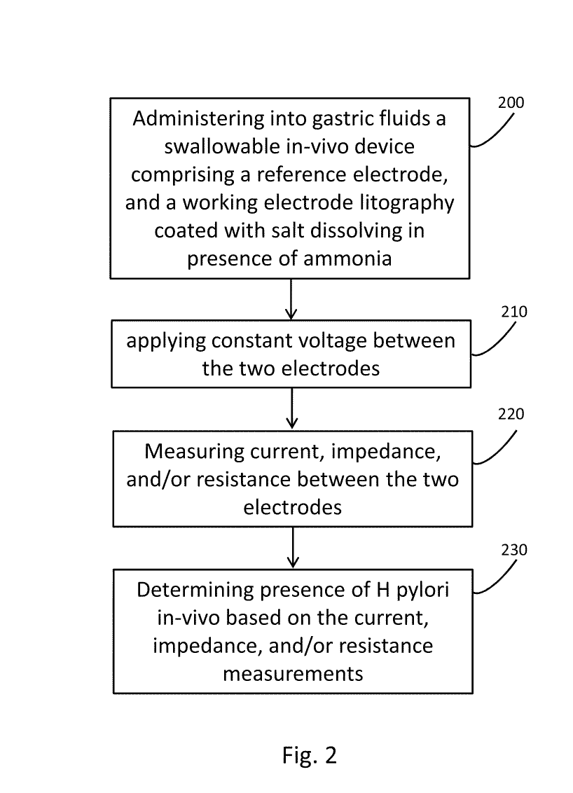

Reference is now made to FIG. 2, which illustrates a method of in-vivo detection of H. pylori, in accordance with one embodiment of the present invention. The method may comprise the step of administering into gastric fluids a swallowable in-vivo device, e.g., device 10, which comprises two electrodes, e.g., reference electrode 12 and working electrode 13 and measuring device 14 (200). The method may further comprise the steps of applying constant voltage between the two electrodes (210), measuring current, impedance, and/or resistance between the two electrodes using the measuring device, e.g., an ampere-meter (220), and determining presence of H. pylori based on the current, impedance, and/or resistance measurements (230). In some embodiments, reference electrode 12 may be made of an inert material, e.g., gold. In some embodiments, working electrode 13, may be made of an inert material, e.g., gold, and substantially entirely coated with a salt dissolvable in the presence of ammonia, e.g., silver-chloride. In other embodiments, working electrode 13 may be coated with silver-chloride mixed with a semi-permeable membrane, which create a semi-permeable coating over electrode 13. In the absence of H. pylori in gastric fluids, the coating on working electrode 13 may remain intact and measured current between reference electrode 12 and working electrode 13 would be approximately zero. However, in the presence of H. pylori, ammonia (which is H. pylori's byproduct) is also present within the gastric fluids. Ammonia may cause the coating on working electrode 13 to dissolve, thus causing working electrode 13 to be in contact with gastric fluids, which leads to current changes between the two electrodes. Typically, since constant voltage is applied in device 10, the current will rise or increase above zero and resistance will thus decrease. These current changes may be measured by measuring device 14, e.g., an ampere-meter, and may be processed by either a processor housed within the in-vivo device, or a processor external to the in-vivo device, so that an indication or determination on presence or absence of H. pylori may be made. In some embodiments, the current measurements may be translated into resistance measurements by a processor, according to equation (i). In other embodiments, an ohmmeter may replace ampere-meter 14, thus resistance may be directly measured instead of current. In the presence of ammonia, since current increases, resistance and impedance decreases, as apparent from equation (i).

This method of measuring current between electrodes 12 and 13 is much more sensitive compared to measuring voltage changes before and after presence of ammonia in in-vivo fluids. When applying constant voltage in the in-vivo device, and prior to presence of ammonia within in-vivo fluids, the current between electrodes 12 and 13 is zero. When there is even a slight change in presence of ammonia, ammonia will begin to dissolve the salt coating electrode 13, and thus create contact between electrode 13 and in-vivo fluids, which will cause current to pass between electrode 13 and electrode 12. Such a change in current, i.e., an increase in current will be noticed by the device, e.g., by a processor, and will indicate on presence of ammonia, thus indicating presence of H. pylori. Even if this change in current may take some time to be noticed, since it takes time until ammonia dissolves the coating covering electrode 13, once the coating has dissolved, the change in current would be immediately noted by measuring device 14, e.g., an ampere-meter. On the contrary, when measuring voltage changes of before and after presence of H. pylori, the changes need to be substantial changes in order for the system to detect them and in order for these changes to be significant and thus include variations between patients. Typically, in order for voltage changes to be significant, due to presence of ammonia, high levels of ammonia should be present within the fluid, which is not necessarily conforming to levels of ammonia in the human body in the event that H. pylori is present inside in-vivo gastric fluids.

In some embodiments, a connection may be found between the rate of current increase and the concentration of ammonia inside the stomach. The rate of increase of current passing between electrodes 12 and 13 may be proportional to ammonia concentration in the stomach. That is, the higher the increase in current measured by the device, the higher the concentration of ammonia present in the gastric lumen. In some embodiments, such a proportion may be normalized into specific numbers, such that a certain increase would indicate on a certain ammonia concentration. Ammonia concentrations may indicate on severity of patient's condition, therefore, it may be beneficial for a physician to have a tool providing such information.

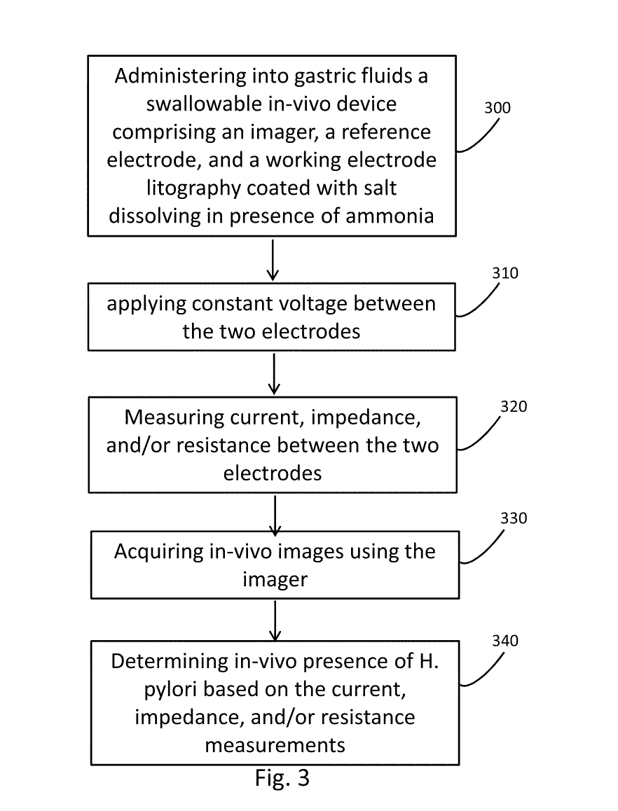

Reference is now made to FIG. 3, which illustrates a method of in-vivo detection of H. pylori, in accordance with an embodiment of the present invention. The method may comprise the step of administering into gastric fluids a swallowable in-vivo device, e.g., device 10, which comprises two electrodes, e.g., reference electrode 12 and working electrode 13, measuring device 14, and in addition an imager (300). The method may further comprise the steps of applying constant voltage between the two electrodes (310), measuring current and/or resistance between the two electrodes using the ampere-meter (320), acquiring in-vivo images using the imager within the swallowable in-vivo device (330), and determining presence of H. pylori based on the current and/or resistance measurements (340). In some embodiments, reference electrode 12 may be made of an inert material, e.g., gold. In some embodiments, working electrode 13, may be made of an inert material, e.g., gold, and substantially entirely coated with a salt dissolvable in the presence of ammonia, e.g., silver-chloride. In other embodiments, working electrode 13 may be coated with silver-chloride mixed with a semi-permeable membrane, which create a semi-permeable coating over electrode 13. In the absence of H. pylori in gastric fluids, the coating on working electrode 13 may remain intact and measured current between reference electrode 12 and working electrode 13 would be approximately zero. However, in the presence of H. pylori, ammonia (which is H. pylori's byproduct) is also present within the gastric fluids. Ammonia may cause the coating on working electrode 13 to dissolve, thus causing working electrode 13 to be in contact with gastric fluids, which leads to current changes between the two electrodes 12 and 13. Typically, since constant voltage is applied in device 10, the current will rise or increase above zero and resistance will thus decrease. These current changes may be measured by measuring device 14, e.g., an ampere-meter, and may be processed by either a processor housed within the in-vivo device, or a processor external to the in-vivo device, so that an indication or determination on presence of H. pylori may be made. In some embodiments, the current measurements may be translated into resistance measurements by a processor, according to equation (i). In other embodiments, an ohmmeter may replace ampere-meter 14, thus resistance may be directly measured instead of current. In the presence of ammonia, since current increases, resistance decreases, as apparent from equation (i). While the device is in-vivo, it may acquire images of the gastric lumen. These images may be used to detect pathologies (e.g., ulcers) that may be a result of presence of ammonia inside the stomach. Such images may be used in combination of the current and/or resistance measurements in order to confirm presence of ammonia inside the stomach, thus confirming presence of H. pylori.

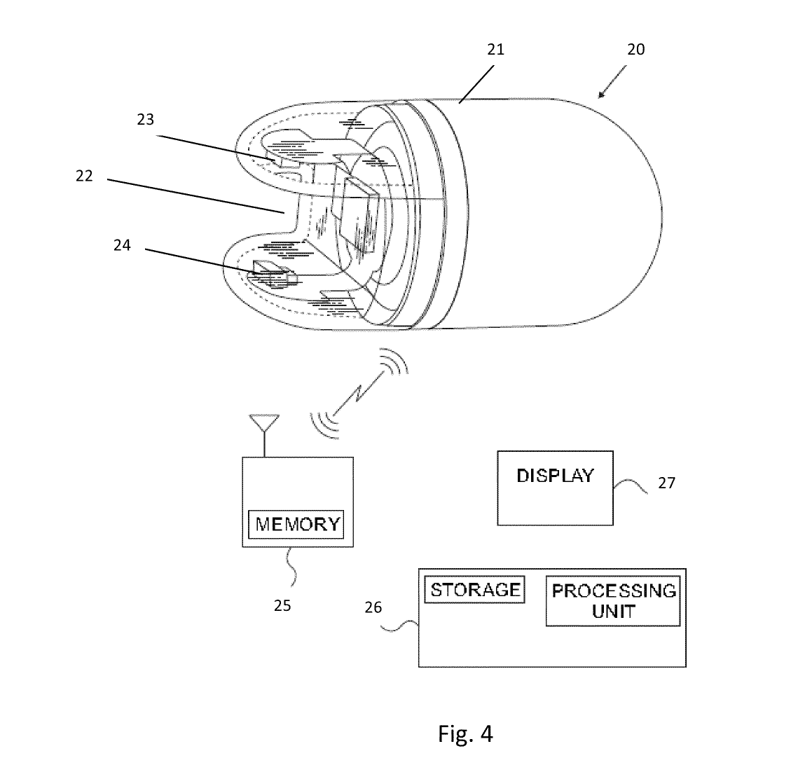

Reference is now made to FIG. 4, which illustrates a schematic side view of an in-vivo device and system, in accordance with an embodiment of the present invention. According to FIG. 4, device 20 may be an autonomous swallowable device and may comprise a housing 21. Housing 21 may have various shapes, which should be easy on a patient to swallow, e.g., spherical, ellipsoidal, etc. On one end of housing 21, there may be a gap 22. On one side of gap 22 there may be a light source 23 (e.g., a LED), and on the opposite side of gap 22, and facing across the gap towards illumination source 23, there may be a detector 24. In other embodiments, light source 23 and detector 24 may be on the same side of gap 22, but a minor or any other kind of reflector should be positioned on the opposite side of gap 22, facing across the gap towards light source 23 and detector 24. Gap 22 may be at least partially filled with a mixture of components such as an enzyme and substrate, some of which change their optical properties in the presence of ammonia. The mixture of components may be held in gap 22 by being applied onto the inner surface of gap 22 in the form of a gel, which may be covered by a permeable coating that enables passage of water, ammonia and other materials typically flowing within in-vivo fluids. The permeable coating enables passage of fluids in and out of the inner surface of gap 22, i.e., in and out of the gel-like mixture of enzyme and substrate. In-vivo fluids may enter and exit gap 22 such that ammonia (if present) may be in contact with the mixture of components located within gap 22, and thus cause at least some of the components to change their optical properties. For example, a change in optical properties may be a change in color of a component or the component may absorb less light of a certain illuminated wavelength following reaction with ammonia, compared to the absorbance of light before reaction with ammonia. In one example, the enzyme may be L-Glutamate Dehydrogenase (GDH), and the substrate may comprise a-ketoglutaric acid (KGA), nicotinamide adenine dinucleotide phosphate (NADPH), buffers, stabilizers, and nonreactive fillers, as may be found in kits provided by SIGMA-ALDRICH®, Mo., USA. The reaction occurring in the presence of ammonia (which indicates presence of H. pylori) is as follows:

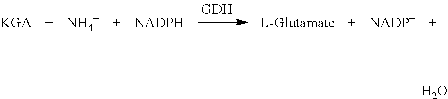 [SMILES: [NH4+] ; O]

Following oxidation of NADPH due to the presence of ammonia, there is a decrease in absorbance in the wavelength of 340 nm. The decrease in absorbance in 340 nm is proportional to the ammonia concentration. That is, detection of light absorbance may indicate on ammonia concentration, which may indicate on H. pylori concentration. The wavelength at which light source 32 may illuminate may be chosen such to correlate with the optical properties of the materials that react with ammonia (which indicates presence of H. pylori). For example, when using materials such as disclosed above, i.e., NADPH, illumination source 32 may be designed to illuminate at a wavelength of 340 nm, though other wavelengths may be used.

Detector 24 may be used to detect changes in optical properties of the substrate, for example, a change in absorbance of light by the substrate within gap 22. The amount of decrease in light absorbance over time by NADPH may be used to determine not only presence of H. pylori but also the concentration of H. pylori. Other materials, which may indicate presence of ammonia and thus presence of H. pylori, may be inserted into gap 22.

According to some embodiments, device 20 may be part of an entire system for the detection of H. pylori. Device 20 may comprise a transmitter used to transmit readings of light absorbance by detector 24 to an external receiver 25. External receiver 25 may comprise a memory unit. The system may further comprise a storage and processing unit 26, which may process the detected light absorbance to determine presence or absence of H. pylori, and/or concentration of ammonia and/or H. pylori. The system may further comprise a display unit 27 for displaying the readings of detector 24. Display unit 27 may also be used to display the processed readings, e.g., determination of presence or absence of H. pylori within gastric fluids of the patient who swallowed device 20, as well as concentration of ammonia and/or H. pylori.

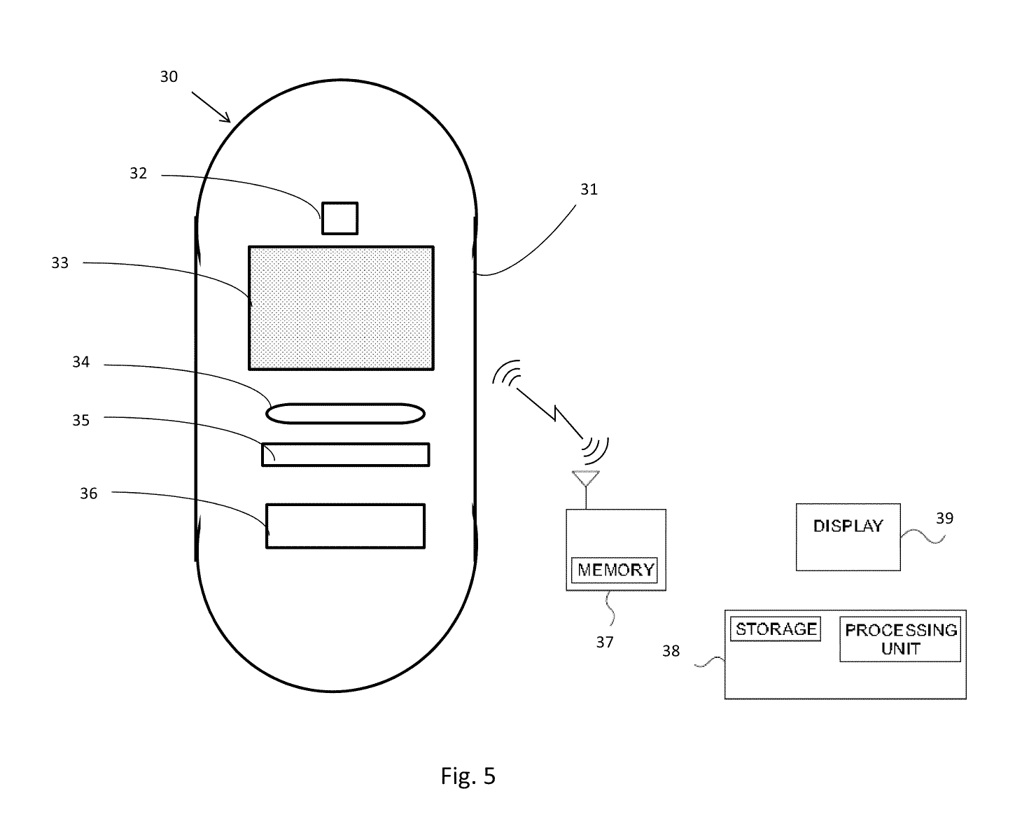

Reference is now made to FIG. 5, which schematically illustrates an in-vivo device and system, in accordance with an embodiment of the present invention. Device 30 may be an autonomous swallowable device. Device 30 may comprise a housing 31, which may have various shapes configured for being swallowed, e.g., capsule, spherical, ellipsoidal, etc. Device 30 may comprise an opening or gap 33 created within one end of housing 31. Gap 33 may be covered by one end of housing 31 and may pass therethrough. On one side of gap 33 there may be illumination source 32, e.g., a LED, and on the opposite side of gap 33, and facing across the gap towards illumination source 32, there may be optical system 34 and detector 35. Optical system 34 may be located between gap 33 and detector 35, such that light irradiated by illumination source 32 may pass through gap 33, and then through optical system 34 in order to be detected by detector 35. Gap 33 may comprise materials similar to the ones used to fill in gap 22 (FIG. 4). Gap 33 may be filled with a mixture of components such as an enzyme and substrate, some of which change their color or absorb less light of a certain illuminated wavelength in the presence of ammonia. For example, the enzyme may be L-Glutamate Dehydrogenase (GDH), and the substrate may comprise a-ketoglutaric acid (KGA), nicotinamide adenine dinucleotide phosphate (NADPH), buffers, stabilizers, and nonreactive fillers, as may be found in kits provided by SIGMA-ALDRICH®, Mo., USA.

The wavelength at which light source 32 may illuminate may be chosen such to correlate with the optical properties of the materials that react with ammonia (which indicates presence of H. pylori). For example, when using materials such as disclosed above, illumination source 32 may be designed to illuminate at a wavelength of 340 nm, though other wavelengths may be used.

If ammonia is present within the gastric fluids, ammonia may be in contact with the substrate and enzyme and may cause a change in optical properties of the substrate. For example, ammonia may cause a change in color or a decrease in absorbance of the substrate, e.g., NADPH. Illumination source 32 may illuminate the in-vivo fluids, which may freely enter and exit gap 33. Light passing through the fluids and the mixture of materials may be collected by optical system 34 and then focused onto detector 35 by optical system 34. Detector 35 may detect the changes in optical properties of the substrate. The readings by detector 35 may be recorded over time, and thus the changes in optical properties, e.g., in absorbance of the light may be recorded over time. Since the decrease in absorbance of NADPH over time is proportional to the concentration of ammonia, determination regarding presence as well as concentration of ammonia and/or H. pylori may be achieved.

Device 30 may further comprise a transmitter 36 for transmitting the readings of detector 35 to an external receiver 37. External receiver 37 may comprise a memory unit, and a processing unit 38 for processing the readings and providing determination on in-vivo presence of H. pylori and/or ammonia, and possibly their concentration within the gastric fluids of the patient. In some embodiments, external receiver 37 may be connected to a display unit 39. Display unit 39 may or may not display the readings of detector 35, and may display determination of presence of ammonia and/or H. pylori, and/or concentration of ammonia and/or H. pylori to an operator performing a procedure for detection of H. pylori using an in-vivo device such as device 30.

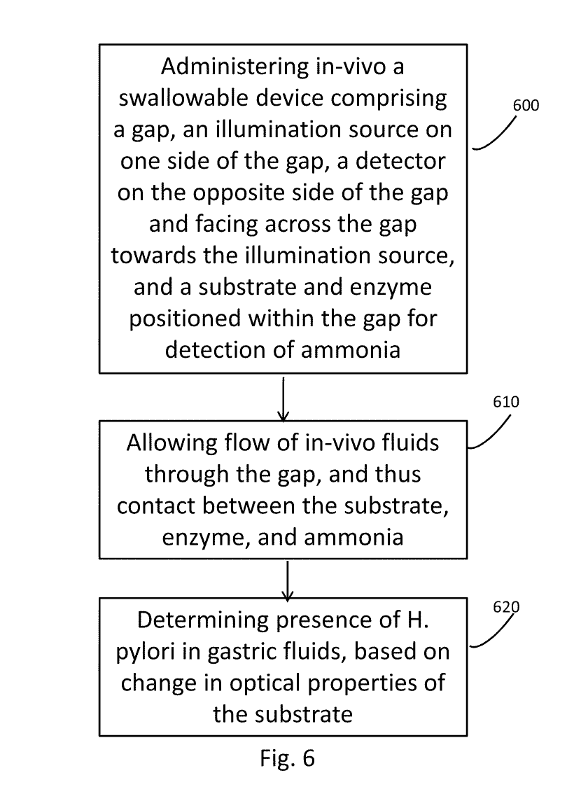

Reference is now made to FIG. 6, which illustrates a method of in-vivo detection of H. pylori, in accordance with an embodiment of the present invention. The method may comprise the following steps: administering in-vivo a swallowable device comprising a gap, an illumination source on one side of the gap, and a detector on the opposite side of the gap and facing across the gap towards the illumination source, and a substrate and enzyme positioned within the gap for detection of ammonia (600). The swallowable device administered by the patient may be, for example, in-vivo device 20 or in-vivo device 30 or any other similar device. The method may further comprise the steps of allowing flow of in-vivo fluids through the gap, and thus contact between the substrate, enzyme and ammonia (610), and determining in-vivo presence of H. pylori, specifically in gastric fluids, based on change in optical properties of the substrate due to presence of ammonia (620). A change in optical properties may, for example, be a change in color or decrease in absorption of light by the substrate due to configurational changes of the substrate caused by reaction of the substrate with ammonia. The change in optical property over time may be proportional to the concentration of ammonia, thus determination on presence of ammonia, and thus on presence of H. pylori may be made along with determination on concentration of ammonia and/or H. pylori.

While the present invention has been described with reference to one or more specific embodiments, the description is intended to be illustrative as a whole and is not to be construed as limiting the invention to the embodiments shown. It is appreciated that various modifications may occur to those skilled in the art that, while not specifically shown herein, are nevertheless within the scope of the invention.

What is claimed is:

1. An in-vivo device for in-vivo detection of H. pylori, said device comprising:
a housing;
a reference electrode disposed on the housing;
a working electrode disposed on the housing in close proximity to said reference electrode, said working electrode coated by litography or screen printing with salt dissolvable in the presence of ammonia such that substantially no current can pass between the working electrode and the reference electrode when coated;
a measuring device for measuring current, impedance and/or resistance between the working and the reference electrodes; and
a transmitter for transmitting said measurements .

2. The in-vivo device according to claim 1, wherein said in-vivo device further comprises a processor for processing the measurements into an indication of presence or absence of H. pylori.

3. The in-vivo device according to claim 1, wherein said measuring device is an ampere-meter.

4. The in-vivo device according to claim 1, wherein said in-vivo device is an autonomous swallowable device.

5. The in-vivo device according to claim 1, wherein said reference electrode is made of gold.

6. The in-vivo device according to claim 1, wherein said working electrode is made of gold.

7. The in-vivo device according to claim 1, wherein said working electrode is litography coated with silver-chloride salt.

8. The in-vivo device according to claim 1, wherein said working electrode is coated by screen printing with silver-chloride salt.

9. A system for in-vivo detection of H. pylori, said system comprising:
the in-vivo device according to claim 1;
a receiver for receiving the transmitted measurements of current, impedance, and/or resistance;
a processing unit for processing the received measurements and providing an indication on presence or absence of H. pylori; and
a display unit for displaying said current, impedance, and/or resistance measurements along with the indication on presence or absence of H. pylori.

10. An in-vivo device for in-vivo detection of H. pylori, said device comprising:
a housing;
a gap through which in-vivo fluids may enter and exit;
a mixture of substrate and enzyme for reacting with ammonia, which causes the substrate to change its optical properties;
an illumination source for illuminating said mixture, said illumination source located on one side of the gap;
a detector for detecting the change in optical properties of the substrate that indicate presence of H. pylori, wherein said detector is located on an opposite side of the gap and facing across the gap towards the illumination source; and
a transmitter for transmitting said detected changes in optical properties that indicate presence of H. pylori.

11. The in-vivo device according to claim 10, wherein said change in optical property of the substrate is a change in light absorbance by the substrate.

12. The in-vivo device according to claim 10, wherein said in-vivo device is an autonomous swallowable device.

13. A system for in-vivo detection of H. pylori, said system comprising:
an in-vivo device according to claim 10;
a receiver for receiving the transmitted changes in optical properties;
a processing unit for processing the received changes in optical properties and determining presence or absence of H. pylori; and
a display unit for displaying said changes in optical properties along with determination of presence or absence of H. pylori.

14. A method for in-vivo detection of H. pylori, said method comprising the following steps:
administering into gastric fluids a swallowable in-vivo device comprising a reference electrode, a working electrode litography coated with salt, and an ampere-meter;
measuring current between the reference and working electrodes using said ampere-meter; and
determining presence of H. pylori in-vivo based on said current measurements.

15. The method according to claim 14, wherein an increase in current measurement between the reference and working electrodes indicates in-vivo presence of H. pylori.

16. A method for in-vivo detection of H. pylori, said method comprising the following steps:
administering in-vivo a swallowable device, said device comprising: a gap, an illumination source located on one side of the gap, a detector located on the opposite side of the gap and facing across the gap towards said illumination source, and a substrate and enzyme located within the gap for detection of ammonia;
allowing flow of in-vivo fluids through the gap, and thus allowing contact between the substrate, enzyme, and ammonia; and
determining in-vivo presence of H. pylori, based on change in optical properties of the substrate.
